(19) United States
(12) Patent Application Publication
(10) Pub. No.: US 2019/0142298 A1
(43) Pub. Date: May 16, 2019

(54) MAGNETIC RESONANCE QUALITY CONTROL METHOD, SERVER AND SYSTEM

(71) Applicant: TAISHAN MEDICAL UNIVERSITY, Taian, Shandong (CN)

(72) Inventors: Kun HOU, Taian (CN); Jianfeng QIU, Taian (CN); Hong WANG, Taian (CN); Liting SHI, Taian (CN); Weizhao LU, Taian (CN)

(73) Assignee: TAISHAN MEDICAL UNIVERSITY, Taian, Shandong (CN)

(21) Appl. No.: 16/308,847

(22) PCT Filed: Jun. 23, 2017
(86) PCT No.: PCT/CN2017/089854

§ 371 Date: Dec. 11, 2018

(30) Foreign Application Priority Data
Jan. 25, 2017 (CN) 201710055768.2

(51) Int. Cl. A61B 5/055; G16H 40/67; G01R 33/48

(52) U.S. Cl. CPC A61B 5/055; G16H 40/67; H04N 17/00; A61B 2560/0271; G01R 33/48

(57) ABSTRACT

A magnetic resonance quality control method, server and system, wherein the method includes: receiving a quality control report set sent by a test terminal, which carries a detection identifier of a to-be-detected magnetic resonance imager and a target parameter; determining a reference parameter according to the detection identifier; obtaining a device quality report by comparing the reference parameter with the target parameter; and sending the device quality report to a corresponding test terminal according to corresponding relationship. The server provides an evaluation report of the imager to the client by analyzing the quality control reports of a large number of magnetic resonance imagers, the performance of the imager can be comprehensively and accurately evaluated, and it is beneficial for the client to understand the performance of the device more accurately and to quickly locate a fault of the imager and perform maintenance.

### FIELD OF THE INVENTION

The present invention relates to the technical field of medical imaging devices, and in particular, to a magnetic resonance quality control method, a server and a system.

### BACKGROUND OF THE INVENTION

A magnetic resonance imaging system uses a static magnetic field and a radio frequency magnetic field to image a human tissue. In the imaging process, a high-contrast clear image can be obtained without ionizing radiation or a contrast agent. It can reflect the abnormality and early lesion of a human organ from the inside of human molecules. In order to ensure the normal operation of a magnetic resonance imaging system, quality control of the magnetic resonance imaging system is an important part of daily maintenance of an imaging device.

At present, quality testing is usually performed on a magnetic resonance imaging device using a mass detection phantom, and a technical person performs calculation and analysis according to test images and data, thereby obtaining a performance parameter of the imaging device. However, the inventor found through research that this manual method involves a long calculation time, complicated analysis and low efficiency; moreover, due to different processing methods of test data, it is susceptible to human measurement error interference, low in precision, and not conducive to the standardization of quality control.

### SUMMARY OF THE INVENTION

The embodiments of the present invention provide a magnetic resonance quality control method, a server and a system, so as to solve the problems of low efficiency and poor precision of magnetic resonance quality control in the prior art.

In order to solve the above problems, the embodiments of the present invention disclose the following technical solutions:

In a first aspect, an embodiment of the present invention provides a magnetic resonance quality control method, the method comprising:

receiving a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;
determining a reference parameter of the to-be-detected magnetic resonance imager according to the detection identifier;
obtaining a device quality report of the to-be-detected magnetic resonance imager by comparing the reference parameter with the target parameter; and sending the device quality report to a corresponding test terminal according to the corresponding relationship so that the test terminal displays the device quality report.

Optionally, determining a reference parameter of the to-be-detected magnetic resonance imager according to the detection identifier of the to-be-detected magnetic resonance imager comprises:

when the detection identifier includes a magnetic resonance quality control phantom type used by the to-be-detected magnetic resonance imager, selecting a corresponding reference parameter according to the magnetic resonance quality control phantom type, wherein the reference parameter includes an AAPM standard parameter and/or ACR standard parameter;
obtaining a device quality report of the to-be-detected magnetic resonance imager by comparing the reference parameter with the target parameter comprises:
comparing the reference parameter with the target parameter to determine whether the target parameter is qualified; and
if the target parameter is unqualified, generating the device quality report according to maintenance information corresponding to the unqualified target parameter.

Optionally, determining a reference parameter of the to-be-detected magnetic resonance imager according to the detection identifier of the to-be-detected magnetic resonance imager comprises:

when the detection identifier includes a device model of the to-be-detected magnetic resonance imager, obtaining the reference parameter from a quality detection report corresponding to the device model;
obtaining a device quality report of the to-be-detected magnetic resonance imager by comparing the reference parameter with the target parameter comprises:
comparing the reference parameter with the target parameter to determine a quality level of the to-be-detected magnetic resonance imager; and
obtaining a device quality report of the to-be-detected magnetic resonance imager according to the quality level.

Optionally, determining a reference parameter of the to-be-detected magnetic resonance imager according to the detection identifier of the to-be-detected magnetic resonance imager comprises:

when the detection identifier includes an identification code of the to-be-detected imager, obtaining a historical quality detection report of the to-be-detected magnetic resonance imager according to the identification code; and obtaining a reference parameter from the historical quality detection report;
obtaining a device quality report of the to-be-detected magnetic resonance imager by comparing the reference parameter with the target parameter comprises:
determining a warning level of the to-be-detected magnetic resonance imager by comparing the reference parameter with the target parameter; and
obtaining a device quality report of the to-be-detected magnetic resonance imager according to the warning level.

Optionally, determining a reference parameter of the to-be-detected magnetic resonance imager according to the detection identifier of the to-be-detected magnetic resonance imager comprises:

when the detection identifier includes a manufacturer of the to-be-detected magnetic resonance imager, obtaining the reference parameter from a quality detection report of a magnetic resonance imager produced by the manufacturer;
obtaining a device quality report of the to-be-detected magnetic resonance imager by comparing the reference parameter with the target parameter comprises:
determining a quality ranking of the to-be-detected magnetic resonance imager by comparing the reference parameter with the target parameter; and
obtaining a device quality report of the to-be-detected magnetic resonance imager according to the quality ranking.

Optionally, the target parameter and the reference parameter respectively include a combination of one or more of spatial resolution, contrast resolution, signal to noise ratio, laser calibration, geometric distortion, and artifact.

In a second aspect, an embodiment of the present invention provides a server for magnetic resonance quality control, the server comprising:

a receiving module configured to receive a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;
a determining module configured to determine a reference parameter of the to-be-detected magnetic resonance imager according to the detection identifier;
a generation module configured to obtain a device quality report of the to-be-detected magnetic resonance imager by comparing the reference parameter with the target parameter; and
a sending module configured to send the device quality report to a corresponding test terminal according to the corresponding relationship so that the test terminal displays the device quality report.

In a third aspect, an embodiment of the present invention further provides a magnetic resonance quality control system, the system comprising a magnetic resonance quality control phantom, a test terminal, and a data server, wherein:

the magnetic resonance quality control phantom is configured to be placed in an imaging center of a to-be-detected magnetic resonance imager, and test the to-be-detected magnetic resonance imager;
the test terminal is communicatively connected to the to-be-detected magnetic resonance imager to acquire scan data of the magnetic resonance quality control phantom by the to-be-detected magnetic resonance imager;
the test terminal is further communicatively connected to the data server for performing data analysis on the scan data to obtain a quality control report, and sending the quality control report to the data server.

Optionally, the system further comprises a user server, wherein:

the user server is communicatively connected to the data server for acquiring a device quality report from the data server;
the user server is further communicatively connected to a user terminal and a manufacturer terminal for sending the device quality report to a corresponding user terminal, and sending a quality control report corresponding to the manufacturer to the manufacturer terminal after receiving a report request sent by the manufacturer terminal.

Optionally, the system further comprises a test terminal server, wherein:

the test terminal server is communicatively connected to a data server for sending a quality control report to the data server, and receiving a device quality report sent by the data server;
the test terminal server is further communicatively connected to at least one test terminal for receiving and storing a quality control report from a test terminal, and sending the device quality report to a corresponding test terminal according to corresponding relationship.

The technical solutions provided in the embodiments of the present disclosure may include the following beneficial effects: the embodiments of the present invention provide a magnetic resonance quality control method, a server and a system. The method comprises: receiving a quality control report set sent by a test terminal, wherein the quality control report set comprises at least one quality control report, and the quality control report set has corresponding relationship with the test terminal; the quality control report at least carrying a detection identifier of a to-be-detected magnetic resonance imager, and a target parameter obtained by testing the to-be-detected magnetic resonance imager; determining a reference parameter of the to-be-detected magnetic resonance imager according to the detection identifier; obtaining a device quality report of the to-be-detected magnetic resonance imager by comparing the reference parameter with the target parameter; and sending the device quality report to a corresponding test terminal according to the corresponding relationship so that the test terminal displays the device quality report. According to the present invention, the quality control report is automatically obtained by the test terminal without the need for manual analysis and calculation by a client, thus saving time and effort, and the client can spend more time on the maintenance and adjustment of the device, thereby greatly improving the efficiency; the server can provide an evaluation report of the imager to the client by analyzing the quality control reports of a large number of magnetic resonance imagers, the performance of the imager can be comprehensively and accurately evaluated, it is beneficial for the client to have a broader understanding of the performance of the imager used by the client and also beneficial for the client to quickly locate a fault of the imager and perform maintenance under the guidance of the evaluation report; and a manufacturer of the magnetic resonance imager can also download data from an online background processing system and provide data support to R&D personnel so as to better develop, improve and optimize the imager.

It should be understood that the above general description and the subsequent detailed description are exemplary and illustrative, and cannot limit the present disclosure.

### BRIEF DESCRIPTION OF THE DRAWINGS

The drawings herein are incorporated into the specification and form part of the specification, illustrating the embodiments in conformity with the present invention and serving to explain the principles of the present invention together with the specification.

In order to more clearly illustrate the embodiments of the present invention or the technical solutions in the prior art, the drawings needed in the description of the embodiments or the prior art will be briefly described below. Obviously, a person of ordinary skill in the art can also obtain other drawings from these drawings without any creative effort.

FIG. 1 is a flow diagram of a magnetic resonance quality control method provided in an embodiment of the present invention.

FIG. 2 is a structural diagram of a server for magnetic resonance quality control provided in an embodiment of the present invention;

FIG. 3 is a structural diagram of a magnetic resonance quality control system provided in an embodiment of the present invention.

FIG. 4 is a structural diagram of another magnetic resonance quality control system provided in an embodiment of the present invention.

FIG. 5 is a structural diagram of still another magnetic resonance quality control system provided in an embodiment of the present invention.

### DETAILED DESCRIPTION OF THE EMBODIMENTS

In order to make a person skilled in the art better understand the technical solutions in the present invention, the technical solutions in the embodiments of the present invention will be clearly and completely described in conjunction with the drawings in the embodiments of the present invention. Obviously, the described embodiments are only a part of the embodiments of the present invention, rather than all of the embodiments. All other embodiments obtained by a person of ordinary skill in the art based on the embodiments of the present invention without any creative effort should fall within the protection scope of the present invention.

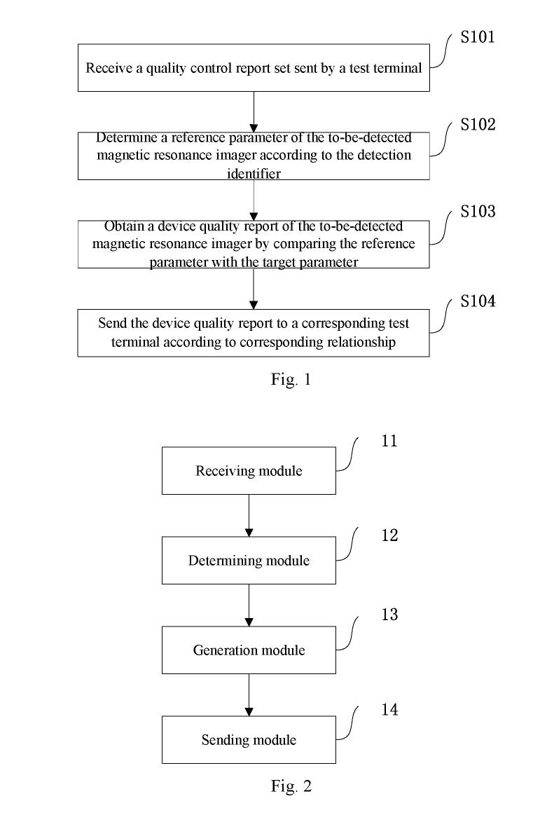

See FIG. 1, which is a flow diagram of a magnetic resonance quality control method provided in an embodiment of the invention. As shown in FIG. 1, an embodiment of the present invention shows a process for performing magnetic resonance quality control by a server:

Step S101: receiving a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;

In order to clearly explain the technical solutions of the embodiments of the present invention, first, the architecture of the test terminal and the server in the embodiments of the present invention are described in detail. The test terminal can be understood as a computer; moreover, in a specific implementation, the test terminal can test an imaging result of a to-be-detected magnetic resonance imager, or can simultaneously test imaging results of a plurality of to-be-detected magnetic resonance imagers to obtain a quality control report of each to-be-detected magnetic resonance imager after the test is completed. One or more test terminals are communicatively connected to the server via the Internet or a local area network, so that the server can receive a quality control report set sent by a test terminal and store the received quality control report set in a database, and the quality control report set at least comprises one quality control report; each quality control report set corresponds to a test terminal in a one-to-one manner, to facilitate the server sending a subsequent processing result to the corresponding test terminal for a user to consult and use.

In order to obtain a quality control report, a dedicated magnetic resonance quality control phantom needs to be placed in the imaging center of the magnetic resonance imager in strict accordance with test instructions, and a dedicated scan sequence and parameters need to be set in strict accordance with the test instructions.

In an exemplary embodiment, an SE (spin echo) sequence may be selected as the dedicated scan sequence, and the parameters may include coil position, TR value, TE value, layer thickness, layer interval, matrix size, and scan starting position, etc. The dedicated scan sequence and parameters set in the exemplary embodiment are shown in Table 1:

TABLE 1

| | | | | Layer | Layer | Matrix | Scan starting |
|---|---|---|---|---|---|---|---|
| Coil | Sequence | TR | TE | thickness | interval | size | position |
| Head coil or | SE (spin echo) | *≥2000 | *≥100 | 3 mm | 0.5 mm | 512 × 512 | Starting layer |
| head and neck | sequence | | | | | | surface or |
| united coil | | | | | | | starting mark |
| | | | | | | | point |

The magnetic resonance imager begins scanning the dedicated magnetic resonance quality control phantom to obtain scan data. The test terminal acquires scan data of the to-be-detected magnetic resonance imager, and automatically processes and analyzes the scan data, obtains a quality control report and presents it to a user.

The quality control report may include test basic information such as device model, manufacturer, production date, test time, dedicated magnetic resonance quality control phantom type, tester name, and the like. The quality control report may include: the scan parameters and sequences used in this scan. These parameters may be read from a header file of the scan data. The quality control report may include a target parameter, and the target parameter includes a combination of one or more of spatial resolution, contrast resolution, signal to noise ratio, uniformity, laser calibration, geometric distortion, and artifact.

In order to facilitate data analysis according to the quality control report, in the embodiments of the present invention, the quality control report further carries a detection identifier of the to-be-detected magnetic resonance imager, and the detection identifier may include a combination of one or more of the magnetic resonance quality control phantom type, device model and manufacturer in the above test basic information, in addition to an identification code for uniquely identifying the to-be-detected magnetic resonance imager.

In this way, by detecting the to-be-detected magnetic resonance imager through the test terminal, a client does not need to manually analyze the scan data, and the precious time of the client is greatly saved, thus freeing the client from the cumbersome data processing to better focus on device adjustment and maintenance. In addition, the quality control report is obtained by the test analysis workstation according to uniform standards and processing methods, which is conducive to the standardization of the quality control report.

Step S102: determining a reference parameter of the to-be-detected magnetic resonance imager according to the detection identifier.

A reference parameter of the to-be-detected magnetic resonance imager is determined according to the detection identifier, and the reference parameter also includes a combination of one or more of spatial resolution, contrast resolution, signal to noise ratio, uniformity, laser calibration, geometric distortion, and artifact, such that the imaging quality of the to-be-detected magnetic resonance imager can be evaluated according to the reference parameter.

In the case of a first implementation, when the detection identifier includes a magnetic resonance quality control phantom type used by the to-be-detected magnetic resonance imager, a corresponding reference parameter is selected according to the magnetic resonance quality control phantom type, wherein the reference parameter includes an AAPM standard parameter and/or ACR standard parameter. In an exemplary embodiment, if the magnetic resonance quality control phantom type in the detection identifier is a quality control phantom of the AAPM standard, the AAPM standard parameter is selected as the reference parameter; if the magnetic resonance quality control phantom type in the detection identifier is a quality control phantom of the ACR standard, the ACR standard parameter is selected as the reference parameter. Of course, in a specific implementation, a combination of the AAPM standard parameter and ACR standard parameter may also be used as the reference parameter.

In the case of a second implementation, when the detection identifier includes a device model of the to-be-detected magnetic resonance imager, the reference parameter is obtained from a quality detection report corresponding to the device model. The server can acquire a device model of the to-be-detected magnetic resonance imager from the detection identifier, and search a database according to the device model, thereby obtaining a quality control report of a magnetic resonance imager that is produced by a same manufacturer or produced by a different manufacturer and is consistent with the device model of the to-be-detected magnetic resonance imager. In an exemplary embodiment, parameters corresponding to target parameters may be extracted from all the retrieved quality control reports, thus forming a parameter group corresponding to each target parameter. All parameter groups further form a parameter set, and the parameter set is used as a reference parameter.

In the case of a third implementation, when the detection identifier includes an identification code of the to-be-detected imager, a historical quality detection report of the to-be-detected magnetic resonance imager is obtained according to the identification code; and a reference parameter is obtained from the historical quality detection report. The server may search a database according to the identification code, thereby obtaining a historical quality detection report of the to-be-detected magnetic resonance imager, and the historical quality detection report can be understood as a quality control report that is obtained by multiple times of detection of the to-be-detected magnetic resonance imager and stored in the database. Parameters corresponding to target parameters are obtained from all the historical detection reports, thus forming a parameter group corresponding to each target parameter. All parameter groups form a parameter set, and the parameter set is used as a reference parameter.

In the case of a fourth implementation, when the detection identifier includes a manufacturer of the to-be-detected magnetic resonance imager, the reference parameter is obtained from a quality detection report of a magnetic resonance imager produced by the manufacturer. The server retrieves a manufacturer from a database, thereby obtaining quality control reports corresponding to all models of magnetic resonance imagers produced by the manufacturer. Parameters corresponding to target parameters are extracted from all the retrieved quality control reports, thus forming a parameter group corresponding to each target parameter. All parameter groups further form a parameter set, and the parameter set is used as a reference parameter.

Step S103: obtaining a device quality report of the to-be-detected magnetic resonance imager by comparing the reference parameter with the target parameter.

An optimized reference parameter is obtained in step S102, and the reference parameter can provide more comprehensive information for quality assessment of the to-be-detected magnetic resonance imager, thereby obtaining a device quality report of the to-be-detected magnetic resonance imager by comparing the reference parameter with the target parameter, and enabling the device quality report to comprehensively and accurately assess the quality of the to-be-detected magnetic resonance imager.

In the case of the first implementation, when the reference parameter includes an AAPM standard parameter and/or ACR standard parameter, it can be determined whether the target parameter is qualified by comparing the reference parameter with the target parameter in a one-to-one manner; and if the target parameter is unqualified, then a qualified target parameter, an unqualified target parameter, and the difference between the unqualified target parameter and the reference parameter may be listed in the device quality report. Moreover, in order to facilitate the overhaul of the to-be-detected magnetic resonance imager, in an embodiment of the present invention, maintenance information corresponding to the unqualified target parameter may be added to the device quality report according to the unqualified target parameter. For example, if the geometric distortion goes beyond an allowable range, a maintenance advice “need to be shimmed” is provided in the device quality report.

In the case of the second implementation, when the reference parameter includes a parameter set of the same device model, in an exemplary embodiment, the target parameter and the reference parameter may be sorted in order of magnitude, thereby determining the position of the target parameter in the parameter set, and then determining the quality level of the to-be-detected magnetic resonance imager according to the position of the target parameter; specifically, when a larger parameter value represents better performance, the target parameter and the reference parameter may be sorted in a descending order, and if the target parameter is located in a front position, it indicates that the target parameter of the to-be-detected magnetic resonance imager is in an excellent range in the same type of magnetic resonance imagers, and it is determined that the quality level of the to-be-detected magnetic resonance imager is excellent; alternatively, if the target parameter is located in a rear position, it indicates that the target parameter of the to-be-detected magnetic resonance imager is in a poor range in the same type of magnetic resonance imagers, and it is determined that the quality level of the to-be-detected magnetic resonance imager is poor. Of course, it should be noted that the method of determining the quality level of the to-be-detected magnetic resonance imager is only an exemplary embodiment. In a specific implementation, the reference parameter may be first optimized, such as determining a plurality of parameter ranges according to the reference parameter, each parameter range corresponding to a corresponding quality level, and determining the quality level of the to-be-detected magnetic resonance imager according to the parameter range to which the target parameter belongs. In addition, in a specific implementation, the quality level may be divided into any number, for example, excellent, good, medium, and poor, according to the test requirements, and is not limited in the embodiments of the present invention. The quality level corresponding to each target parameter is added to the device quality report for a user to evaluate.

In the case of the third implementation, when the reference parameter includes a parameter set extracted in the historical quality detection report, a warning level of the to-be-detected magnetic resonance imager is obtained by comparing the reference parameter with the target parameter. In an exemplary embodiment, a maximum parameter, a minimum parameter, and an average value corresponding to each parameter group may be extracted from the parameter set for each parameter group. For example, when a larger parameter corresponds to better performance, the process of determining the warning level is described in detail; when the target parameter is greater than the corresponding maximum parameter, it indicates that the target parameter of the to-be-detected magnetic resonance imager is excellent in performance, and the lowest warning level is set; when the target parameter is between the maximum parameter and the average value, it indicates that the target parameter of the to-be-detected magnetic resonance imager is better in performance, and a lower warning level is set; when the target parameter is between the average value and the minimum parameter, it indicates that the target parameter of the to-be-detected magnetic resonance imager is lower than the average value in performance, and a higher warning level is set to remind a user that maintenance needs to be performed with respect to the corresponding target parameter; when the target parameter is smaller than the minimum parameter, it indicates that the target parameter of the to-be-detected magnetic resonance imager is the worst in the historical data, and a highest warning level is set to warn the user that maintenance must be performed immediately with respect to the corresponding target parameter. In another exemplary embodiment, a trend graph of performance parameters of the imager over time may be generated according to the historical quality control report of the imager, and fed back to the client, so that the client has a better understanding of the imager in use, thereby performing targeted maintenance on the imager, and it will not be repeated in the embodiment of the present invention; moreover, according to the trend graph, the warning level of the to-be-detected magnetic resonance imager may be further determined, for example, if the trend is to a direction of bad performance, a higher warning level is set, and if the trend is to a direction of good performance, a low early warning may be set, and so on. In addition, it should be noted that the above warning level may include any number of levels, which is not limited in the embodiments of the present invention. The warning level of each target parameter is added to the device quality report, thereby facilitating the user to analyze and determine the performance change trend according to the history record of the to-be-detected magnetic resonance imager, and then formulate a maintenance strategy.

In the case of the fourth implementation, when the reference parameter includes a parameter set of the magnetic resonance imager of the same manufacturer, a quality ranking of the to-be-detected magnetic resonance imager is determined by comparing the reference parameter with the target parameter. In an exemplary embodiment, the quality ranking of the to-be-detected magnetic resonance imager among all models of magnetic resonance imagers of the manufacturer can be determined by ranking the target parameter and the reference parameter in an order of performance from excellent to poor. In another exemplary embodiment, a test pass rate for a certain parameter of the same manufacturer's magnetic resonance imager is counted and fed back to the client; and then according to the test pass rate, a quality ranking of the to-be-detected magnetic resonance imager is obtained by ranking the test pass rate from highest to lowest. The quality ranking of each target parameter is added to the device quality report, thereby facilitating the user to evaluate the quality ranking of the to-be-detected magnetic resonance imager among all of the manufacturer's magnetic resonance imagers.

Step S104: sending the device quality report to a corresponding test terminal according to the corresponding relationship so that the test terminal displays the device quality report.

The server sends the generated device quality report to a corresponding test terminal, so that the device quality report is displayed on the test terminal, and the user can view the device quality report on the test terminal, thereby evaluating an imaging quality of the to-be-detected magnetic resonance imager, and determining a maintenance strategy. Moreover, in order to ensure that the device quality report is accurately sent to the corresponding test terminal, in the specific implementation, the test terminal can put its own identification stamp in each generated quality control report, establish corresponding relationship between the quality control report and the test terminal, and the server then determines to send the generated device quality report to the test terminal for display according to the identification stamp.

Moreover, in order to facilitate the user to view the device quality report, the server can also send the device quality report to a user terminal, which can be understood as a mobile terminal such as a user's mobile phone, PAD or notebook; the server can establish corresponding relationship between the user terminal and the to-be-detected magnetic resonance imager by means of registration, thereby sending the corresponding device quality report to the user terminal for display when the user sends a request through a user terminal.

In addition, in order to facilitate a manufacturer of the magnetic resonance imager to acquire a corresponding quality control report, the server is also connected to a manufacturer terminal, and the server sends a quality control report corresponding to the manufacturer to the manufacturer terminal when receiving a request from the manufacturer.

As can be seen from the description of the above embodiments, the embodiments of the present invention provide a magnetic resonance quality control method. The method comprises: receiving a quality control report set sent by a test terminal, wherein the quality control report set comprises at least one quality control report, and the quality control report set has corresponding relationship with the test terminal; the quality control report at least carrying a detection identifier of a to-be-detected magnetic resonance imager, and a target parameter obtained by testing the to-be-detected magnetic resonance imager; determining a reference parameter of the to-be-detected magnetic resonance imager according to the detection identifier; obtaining a device quality report of the to-be-detected magnetic resonance imager by comparing the reference parameter with the target parameter; and sending the device quality report to a corresponding test terminal according to the corresponding relationship so that the test terminal displays the device quality report. According to the present invention, the quality control report is automatically obtained by the test terminal without the need for manual analysis and calculation by a client thus saving time and effort, and the client can spend more time on the maintenance and adjustment of the device, thereby greatly improving the efficiency; the server can provide an evaluation report of the imager to the client by analyzing the quality control reports of a large number of magnetic resonance imagers, the performance of the imager can be comprehensively and accurately evaluated, it is beneficial for the client to have a broader understanding of the performance of the imager used by the client and also beneficial for the client to quickly locate a fault of the imager and perform maintenance under the guidance of the evaluation report; and a manufacturer of the magnetic resonance imager can also download data from an online background processing system and provide data support to R&D personnel so as to better develop, improve and optimize the imager.

Through the description of the above method embodiments, a person skilled in the art can clearly understand that the present invention can be implemented by means of software plus a necessary general hardware platform. Of course, hardware can also be used, but the former is better in many cases. Based on such understanding, the technical solutions of the present invention in essence, or the portion contributing to the prior art, can be embodied in the form of a software product. The computer software product is stored in a storage medium and includes some instructions for causing a computer device (which may be a personal computer, server, or network device, etc.) to perform all or part of the steps of the methods described in the embodiments of the present invention. The above storage medium includes various types of media that can store program codes, such as a read only memory (ROM), a random access memory (RAM), a magnetic disk, or an optical disk.

Corresponding to a magnetic resonance quality control method embodiment provided in the present invention, the present invention also provides a server for magnetic resonance quality control.

See FIG. 2, which is a structural diagram of a server for magnetic resonance quality control provided in an embodiment of the present invention. The server comprises: a receiving module 11 configured to receive a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;

a determining module 12 configured to determine a reference parameter of the to-be-detected magnetic resonance imager according to the detection identifier;
a generation module 13 configured to obtain a device quality report of the to-be-detected magnetic resonance imager by comparing the reference parameter with the target parameter; and
a sending module 14 configured to send the device quality report to a corresponding test terminal according to the corresponding relationship.

In the case of a first implementation, when the detection identifier includes a magnetic resonance quality control phantom type used by the to-be-detected magnetic resonance imager, the determining module 12 is configured to select a corresponding reference parameter according to the magnetic resonance quality control phantom type, wherein the reference parameter includes an AAPM standard parameter and/or ACR standard parameter; the generation module 13 is configured to compare the reference parameter with the target parameter to determine whether the target parameter is qualified; if the target parameter is unqualified, the device quality report is generated according to maintenance information corresponding to the target parameter.

In the case of a second implementation, when the detection identifier includes a device model of the to-be-detected magnetic resonance imager, the determining module 12 is configured to obtain the reference parameter from a quality detection report corresponding to the device model; the generation module 13 is configured to determine a quality ranking of the to-be-detected magnetic resonance imager by comparing the reference parameter with the target parameter, and obtain a device quality report of the to-be-detected magnetic resonance imager according to the quality ranking.

In the case of a third implementation, when the detection identifier includes an identification code of the to-be-detected imager, the determining module 12 is configured to obtain a historical quality detection report of the to-be-detected magnetic resonance imager according to the identification code, and obtain a reference parameter from the historical quality detection report; the generation module 13 is configured to determine a warning level of the to-be-detected magnetic resonance imager by comparing the reference parameter with the target parameter, and obtain a device quality report of the to-be-detected magnetic resonance imager according to the warning level.

In the case of a fourth implementation, when the detection identifier includes a manufacturer of the to-be-detected magnetic resonance imager, the determining module 12 is configured to obtain the reference parameter from a quality detection report of a magnetic resonance imager produced by the manufacturer; the generation module 13 is configured to determine a quality ranking of the to-be-detected magnetic resonance imager by comparing the reference parameter with the target parameter, and obtain a device quality report of the to-be-detected magnetic resonance imager according to the quality ranking.

See FIG. 3, which is a structural diagram of a magnetic resonance quality control system provided in an embodiment of the present invention. As shown in FIG. 3, the system comprises a magnetic resonance quality control phantom 21, a test terminal 22, and a data server 23, wherein:

the magnetic resonance quality control phantom 21 is configured to be placed in an imaging center of a to-be-detected magnetic resonance imager, and test the to-be-detected magnetic resonance imager;
the test terminal 22 is communicatively connected to the to-be-detected magnetic resonance imager to acquire scan data of the magnetic resonance quality control phantom by the to-be-detected magnetic resonance imager; the test terminal is communicatively connected to the data server; and
the data server 23 is communicatively connected to the test terminal 22 for receiving a quality control report obtained by analyzing the scan data by the test terminal 22, obtaining a device quality report according to the quality control report, and sending the quality control report to the test terminal 22.

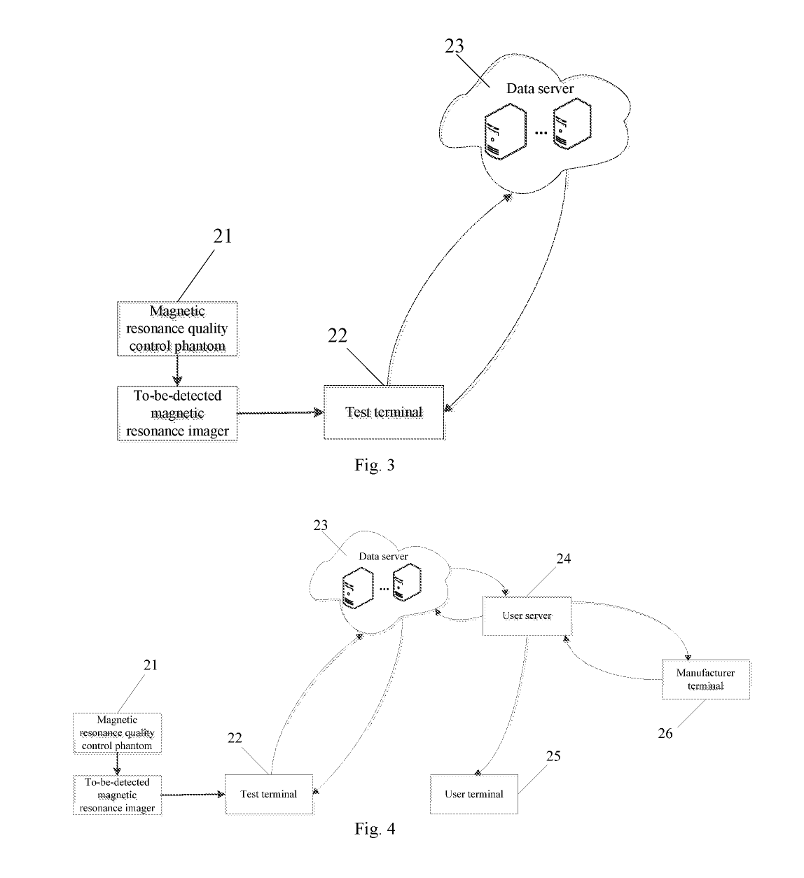

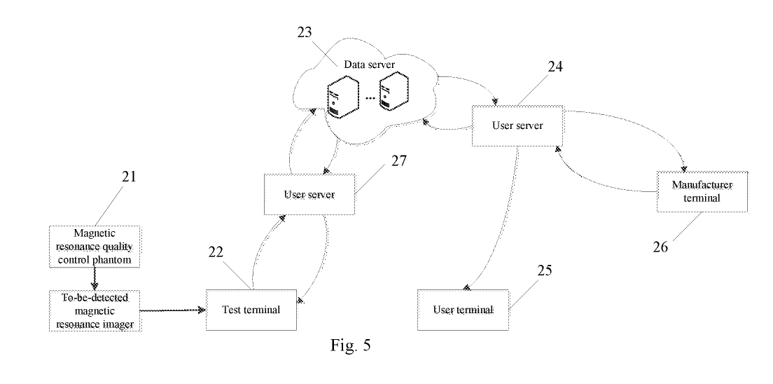

See FIG. 4, which is a structural diagram of another magnetic resonance quality control system provided in an embodiment of the present invention. In order to facilitate information centralized processing and improve information forwarding efficiency, the magnetic resonance quality control system provided in an embodiment of the present invention further comprises a user server 24, wherein:

the user server 24 is communicatively connected to the data server 23 for acquiring a device quality report from the data server;
the user server 24 is further communicatively connected to a user terminal 25 and a manufacturer terminal 26 for sending the device quality report to the user terminal, and sending a quality control report corresponding to the manufacturer to the manufacturer terminal after receiving a report request sent by the manufacturer terminal.

In a specific implementation, the system comprises a plurality of user servers 24, and the plurality of user servers 24 are communicatively connected to each other by cables. See FIG. 5, which is still another structural diagram of a magnetic resonance quality control system provided in an embodiment of the present invention. When a large number of test terminals need to be connected to the data server, in order to facilitate the expansion of the test terminals and the communication efficiency between the test terminals and the data server, in an embodiment of the present invention, the system comprises a test terminal server 27, wherein:

the test terminal server 27 is communicatively connected to a data server 23 for sending a quality control report to the data server, and receiving a device quality report sent by the data server;
the test terminal server 27 is further communicatively connected to at least one test terminal 22 for receiving and storing a quality control report from a test terminal, and sending the device quality report to a corresponding test terminal 22 according to corresponding relationship.

In a specific implementation, the system includes a plurality of test terminal servers 27, and the plurality of test terminal servers 27 are communicatively connected to each other by cables. The test terminal servers 27 and the test terminals 22 are in wireless communication connection by one or more means of the WIFI, 3G/4G and GPRS.

For the convenience of description, the above devices are described as being separately divided into units by function. Of course, the functions of the units may be implemented in one or more software and/or hardware in implementing the present invention.

The present invention may be described in the general context of computer-executable instructions executed by a computer, such as a program module. Generally, the program module includes a routine, program, object, component, data structure, and the like that performs a particular task or implements a particular abstract data type. The present invention may also be practiced in distributed computing environments where tasks are performed by remote processing devices that are connected through a communication network. In a distributed computing environment, a program module may be located in both local and remote computer storage media including a storage device.

Only the specific embodiments of the present invention are described above, enabling a person skilled in the art to understand or implement the present invention. Various modifications to the embodiments will be obvious to those skilled in the art, and the general principles defined herein may be implemented in other embodiments without departing from the spirit or scope of the present invention. Therefore, the present invention will not be limited to the embodiments shown herein, but will conform to the widest range consistent with the principles and novel features disclosed herein.

1. A magnetic resonance quality control method applied to a server, the method comprising the following steps:
receiving a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;
determining a reference parameter of the to-be-detected magnetic resonance imager according to the detection identifier;
obtaining a device quality report of the to-be-detected magnetic resonance imager by comparing the reference parameter with the target parameter; and
sending the device quality report to a corresponding test terminal according to the corresponding relationship so that the test terminal displays the device quality report.

2. The magnetic resonance quality control method according to claim 1, wherein:
determining a reference parameter of the to-be-detected magnetic resonance imager according to the detection identifier of the to-be-detected magnetic resonance imager comprises:
when the detection identifier includes a magnetic resonance quality control phantom type used by the to-be-detected magnetic resonance imager, selecting a corresponding reference parameter according to the magnetic resonance quality control phantom type, wherein the reference parameter includes an AAPM standard parameter and/or ACR standard parameter;
obtaining a device quality report of the to-be-detected magnetic resonance imager by comparing the reference parameter with the target parameter comprises:
comparing the reference parameter with the target parameter to determine whether the target parameter is qualified; and
if the target parameter is unqualified, generating the device quality report according to maintenance information corresponding to the unqualified target parameter.

3. The magnetic resonance quality control method according to claim 1, wherein:
determining a reference parameter of the to-be-detected magnetic resonance imager according to the detection identifier of the to-be-detected magnetic resonance imager comprises:
when the detection identifier includes a device model of the to-be-detected magnetic resonance imager, obtaining the reference parameter from a quality detection report corresponding to the device model;
obtaining a device quality report of the to-be-detected magnetic resonance imager by comparing the reference parameter with the target parameter comprises:
comparing the reference parameter with the target parameter to determine a quality level of the to-be-detected magnetic resonance imager; and
obtaining a device quality report of the to-be-detected magnetic resonance imager according to the quality level.

4. The magnetic resonance quality control method according to claim 1, wherein:
determining a reference parameter of the to-be-detected magnetic resonance imager according to the detection identifier of the to-be-detected magnetic resonance imager comprises:
when the detection identifier includes an identification code of the to-be-detected imager, obtaining a historical quality detection report of the to-be-detected magnetic resonance imager according to the identification code; and obtaining a reference parameter from the historical quality detection report;
obtaining a device quality report of the to-be-detected magnetic resonance imager by comparing the reference parameter with the target parameter comprises:
determining a warning level of the to-be-detected magnetic resonance imager by comparing the reference parameter with the target parameter; and
obtaining a device quality report of the to-be-detected magnetic resonance imager according to the warning level.

5. The magnetic resonance quality control method according to claim 1, wherein:
determining a reference parameter of the to-be-detected magnetic resonance imager according to the detection identifier of the to-be-detected magnetic resonance imager comprises:
when the detection identifier includes a manufacturer of the to-be-detected magnetic resonance imager, obtaining the reference parameter from a quality detection report of a magnetic resonance imager produced by the manufacturer;
obtaining a device quality report of the to-be-detected magnetic resonance imager by comparing the reference parameter with the target parameter comprises:
determining a quality ranking of the to-be-detected magnetic resonance imager by comparing the reference parameter with the target parameter; and
obtaining a device quality report of the to-be-detected magnetic resonance imager according to the quality ranking.

6. The magnetic resonance quality control method according to claim 1, wherein the target parameter and the reference parameter respectively include a combination of one or more of spatial resolution, contrast resolution, signal to noise ratio, uniformity, laser calibration, geometric distortion, and artifact.

7. A server for magnetic resonance quality control, the server comprising:
a receiving module configured to receive a quality control report set sent by a test terminal, wherein the quality control report set comprises at least one quality control report, and the quality control report set has corresponding relationship with the test terminal; the quality control report at least carries a detection identifier of a to-be-detected magnetic resonance imager, and a target parameter obtained by testing the to-be-detected magnetic resonance imager;
a determining module configured to determine a reference parameter of the to-be-detected magnetic resonance imager according to the detection identifier;
a generation module configured to obtain a device quality report of the to-be-detected magnetic resonance imager by comparing the reference parameter with the target parameter; and
a sending module configured to send the device quality report to a corresponding test terminal according to the corresponding relationship so that the test terminal displays the device quality report.

8. A magnetic resonance quality control system, comprising a magnetic resonance quality control phantom, a test terminal, and a data server, wherein:
the magnetic resonance quality control phantom is configured to be placed in an imaging center of a to-be-detected magnetic resonance imager, and test the to-be-detected magnetic resonance imager;
the test terminal is communicatively connected to the to-be-detected magnetic resonance imager to acquire scan data of the magnetic resonance quality control phantom by the to-be-detected magnetic resonance imager; and
the data server is communicatively connected to the test terminal for receiving a quality control report obtained by analyzing the scan data by the test terminal, obtaining a device quality report according to the quality control report, and sending the quality control report to the test terminal.

9. The magnetic resonance quality control system according to claim 8, wherein the system further comprises a user server, wherein:
the user server is communicatively connected to the data server for acquiring a device quality report from the data server;
the user server is further communicatively connected to a user terminal and a manufacturer terminal for sending the device quality report to the user terminal, and sending a quality control report corresponding to the manufacturer terminal to the manufacturer terminal after receiving a report request sent by the manufacturer terminal.

10. The magnetic resonance quality control system according to claim 8, wherein the system further comprises a test terminal server, wherein:
the test terminal server is communicatively connected to a data server for sending a quality control report to the data server, and receiving a device quality report sent by the data server; and
the test terminal server is further communicatively connected to at least one test terminal for receiving and storing a quality control report from a test terminal, and sending the device quality report to a corresponding test terminal according to corresponding relationship.

11. The magnetic resonance quality control system according to claim 9, wherein the system further comprises a test terminal server, wherein:
the test terminal server is communicatively connected to a data server for sending a quality control report to the data server, and receiving a device quality report sent by the data server; and
the test terminal server is further communicatively connected to at least one test terminal for receiving and storing a quality control report from a test terminal, and sending the device quality report to a corresponding test terminal according to corresponding relationship.
